(19) United States
(12) Patent Application Publication
(10) Pub. No.: US 2007/0255138 A1
(43) Pub. Date: Nov. 1, 2007

(54) Method and apparatus for 3D visualization of flow jets

(75) Inventors: Kjell Kristofferson, Oslo (NO); Sevald Berg, Oslo (NO); Andreas Michael Ziegler, Garching (DE)

Correspondence Address: DEAN D. SMALL;THE SMALL PATENT LAW GROUP LLP, 611 OLIVE STREET, SUITE 1611, ST. LOUIS, MO, 63101, US

(73) Assignee: GENERAL ELECTRIC COMPANY

(21) Appl. No.: 11/418,604

(22) Filed: May 5, 2006

Related U.S. Application Data
(60) Provisional application No. 60/795,550, filed on Apr. 27, 2006.

(51) Int. Cl. A61B 8/00

(52) U.S. Cl. 600/443; 600/437

(57) ABSTRACT

A method and apparatus for calculating flow transparency values for voxels representing blood flow within an ultrasonic volume of data comprises identifying velocity values and variance values for voxels within a volume of data. Flow transparency values are calculated for the voxels based on a relationship between the variance value and the velocity value.

### CROSS REFERENCE TO RELATED APPLICATIONS

The application relates to and claims priority from provisional patent application having Ser. No. 60/795,550, entitled “Method and Apparatus for Calculating a Flow Transparancy Value”, filed Apr. 27, 2006, the complete subject matter of which is expressly hereby incorporated herein in its entirety.

### BACKGROUND OF THE INVENTION

This invention relates generally to diagnostic ultrasound systems, and more particularly, to calculating flow transparency values for voxels representative of blood flow.

Diagnosing and assessing turbulent blood flow through a vessel can be challenging with currently available ultrasound systems. Normal blood flow may obscure or otherwise make it difficult to visualize part or all of a turbulent flow jet when displayed on a display or monitor. The blood flow jets that occur within the heart during mitral valve or tricuspid valve regurgitations typically have velocities higher than the Nyquist velocity when using color Doppler. Due to aliasing, high velocity blood flow may be detected and displayed incorrectly as low velocity, and thus not accurately measured.

Volume rendering is used to visualize a set of successive voxels in one direction that have an intensity value and a transparency value (or opacity value). The transparency value is used to determine how much light remains and how much light is reflected from the voxel. In other words, the transparency value determines how opaque or how translucent the voxel is when displayed.

Therefore, a need exists for calculating the flow transparency value to better display areas of turbulent blood flow. Certain embodiments of the present invention are intended to meet these needs and other objectives that will become apparent from the description and drawings set forth below.

### BRIEF DESCRIPTION OF THE INVENTION

In accordance with an embodiment of the present invention, a method for calculating flow transparency values for voxels representing blood flow within an ultrasonic volume of data comprises identifying velocity values and variance values for voxels within a volume of data. Flow transparency values for the voxels are calculated based on a relationship between the variance value and the velocity value.

In accordance with another embodiment of the present invention, an apparatus for displaying blood flow within a volume of ultrasonic data comprises a processor for identifying a variance value and a velocity value for each voxel within a volume of data comprising blood flow. The processor calculates a flow transparency value for each of the voxels based on a relationship between the variance value and the velocity value. A volume rendering processor utilizes the flow transparency values while volume rendering the volume of data. A display displays volume rendered data based on the flow transparency values.

In accordance with another embodiment of the present invention, a method for calculating flow transparency values for voxels representing blood flow within an ultrasonic volume of data comprises identifying a variance value for each of the voxels within a volume of data. The variance value for each of the voxels is compared to a continuum of threshold variance levels. A flow transparency value is calculated for each of the voxels with a transfer function based on at least the variance value and a relationship between the variance value and the continuum of threshold variance levels.

### BRIEF DESCRIPTION OF THE DRAWINGS

FIG. 1 illustrates a block diagram of an ultrasound system formed in accordance with an embodiment of the present invention.

FIG. 2 illustrates an ultrasound system formed in accordance with an embodiment of the present invention.

FIG. 3 illustrates a method for calculating flow transparency values for flow voxels within a volume of ultrasonic data in accordance with an embodiment of the present invention.

FIG. 4 illustrates an alternative method for calculating flow transparency values for flow voxels within a volume of ultrasonic data in accordance with an embodiment of the present invention.

FIG. 5 illustrates a flow chart of a method for flow volume rendering in accordance with an embodiment of the present invention.

FIG. 6 illustrates ultrasound images of two different flow jets displayed with different levels of transparency in accordance with an embodiment of the present invention.

The foregoing summary, as well as the following detailed description of certain embodiments of the present invention, will be better understood when read in conjunction with the appended drawings. The figures illustrate diagrams of the functional blocks of various embodiments. The functional blocks are not necessarily indicative of the division between hardware circuitry. Thus, for example, one or more of the functional blocks (e.g., processors or memories) may be implemented in a single piece of hardware (e.g., a general purpose processor or a block or random access memory, hard disk, or the like). Similarly, the programs may be stand alone programs, may be incorporated as subroutines in an operating system, may be functions in an installed imaging software package, and the like. It should be understood that the various embodiments are not limited to the arrangements and instrumentality shown in the drawings.

### DETAILED DESCRIPTION OF THE INVENTION

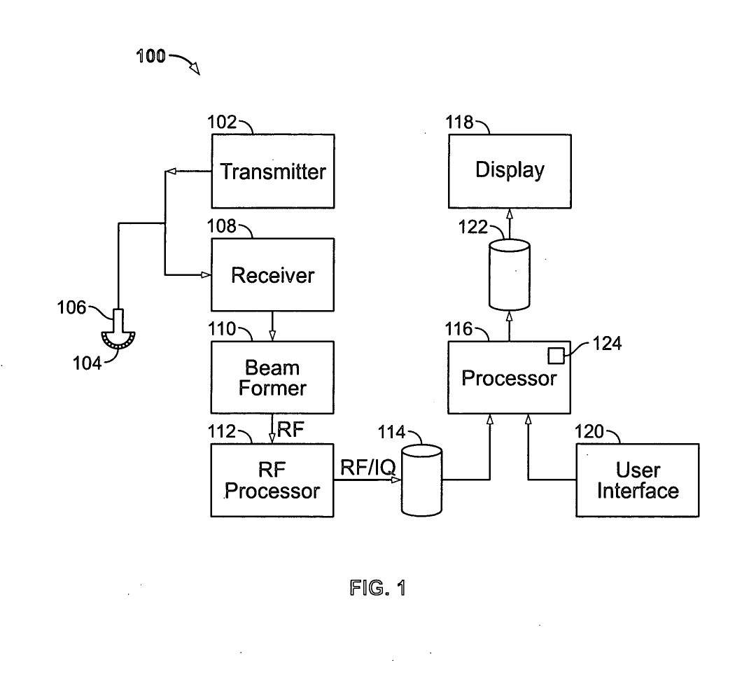

FIG. 1 illustrates a block diagram of an ultrasound system 100. The ultrasound system 100 includes a transmitter 102 which drives transducers 104 within a probe 106 to emit pulsed ultrasonic signals into a body. A variety of geometries may be used. For example, the probe 106 may be used to acquire 2D, 3D, or 4D ultrasonic data, and may have further capabilities such as 3D beam steering. Other types of probes 106 may be used. The ultrasonic signals are back-scattered from structures in the body, like blood cells or muscular tissue, to produce echoes which return to the transducers 104. The echoes are received by a receiver 108. The received echoes are passed through a beamformer 110, which performs beamforming and outputs an RF signal. The beamformer may also process 2D, 3D and 4D ultrasonic data. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to RF/IQ buffer 114 for temporary storage.

The ultrasound system 100 also includes a processor 116 to process the acquired ultrasound information (i.e., RF signal data or IQ data pairs) and prepare frames of ultrasound information for display on display 118. The processor 116 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in RF/IQ buffer 114 during a scanning session and processed in less than real-time in a live or off-line operation. A user interface 120 allows an operator to enter data, enter and change scanning parameters, access protocols, measure structures of interest, and the like. The user interface 120 may be a rotating knob, switch, keyboard keys, mouse, touchscreen, light pen, or any other interface device or method known in the art.

The ultrasound system 100 may continuously acquire ultrasound information at a frame rate that exceeds 50 frames per second—the approximate perception rate of the human eye. The acquired ultrasound information is displayed on the display 118. The ultrasound information may be displayed as B-mode images, M-mode, volumes of data (3D), volumes of data over time (4D), or other desired representation. The operator may use the user interface 120 to input a value to a flow transparency adjustment module 124 to adjust the level of transparency applied to at least portions of a displayed image as discussed below.

An image buffer 122 is included for storing processed frames of acquired ultrasound information that are not scheduled to be displayed immediately. Preferably, the image buffer 122 is of sufficient capacity to store at least several seconds worth of frames of ultrasound information. The frames of ultrasound information are stored in a manner to facilitate retrieval thereof according to its order or time of acquisition. The image buffer 122 may comprise any known data storage medium.

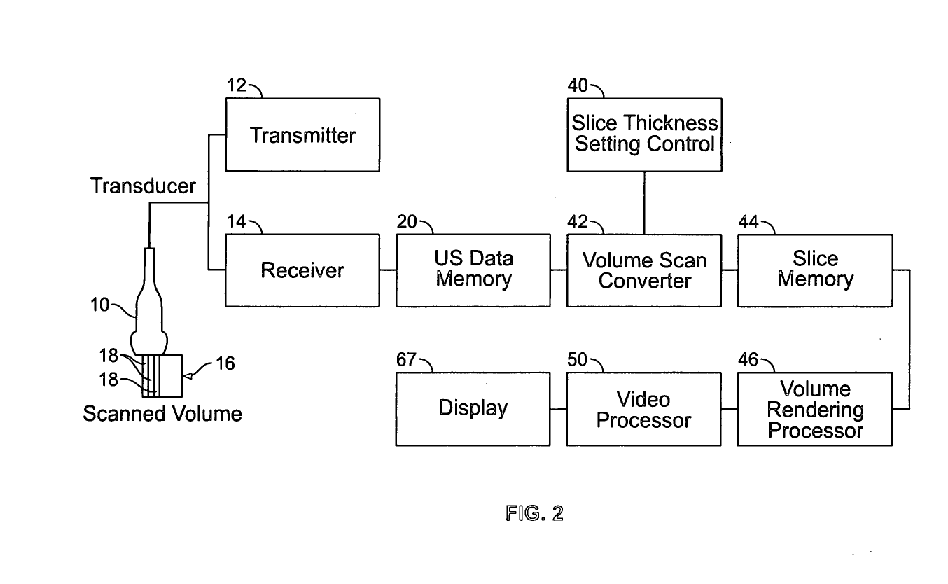

FIG. 2 illustrates an alternative ultrasound system. The system includes a probe 10 connected to a transmitter 12 and a receiver 14. The probe 10 transmits ultrasonic pulses and receives echoes from structures inside of a scanned ultrasound volume 16. Memory 20 stores ultrasound data from the receiver 14 derived from the scanned ultrasound volume 16. The volume 16 may be obtained by various techniques (e.g. 3D scanning, real-time 3D imaging or 4D scanning, volume scanning, 2D scanning with transducers having positioning sensors, freehand scanning using a Voxel correlation technique, 2D or matrix array transducers and the like).

The probe 10 may be moved, such as along a linear or arcuate path, while scanning a region of interest (ROI). At each linear or arcuate position, the probe 10 obtains scan planes 18. The scan planes 18 are collected for a thickness, such as from a group or set of adjacent scan planes 18. The scan planes 18 are stored in the memory 20, and then passed to a volume scan converter 42. In some embodiments, the probe 10 may obtain lines instead of the scan planes 18, and the memory 20 may store lines obtained by the probe 10 rather than the scan planes 18. The volume scan converter 42 may store lines obtained by the probe 10 rather than the scan planes 18. The volume scan converter 42 receives a slice thickness setting from a slice thickness setting control 40, which identifies the thickness of a slice to be created from the scan planes 18. The volume scan converter 42 creates a data slice from multiple adjacent scan planes 18. The number of adjacent scan planes 18 that are obtained to form each data slice is dependent upon the thickness selected by slice thickness setting control 40. The data slice is stored in slice memory 44 and is accessed by a volume rendering processor 46. The volume rendering processor 46 performs volume rendering upon the data slice. The output of the volume rendering processor 46 is passed to the video processor 50 and display 67.

The position of each echo signal sample (Voxel) is defined in terms of geometrical accuracy (i.e., the distance from one Voxel to the next) and ultrasonic response (and derived values from the ultrasonic response). Suitable ultrasonic responses include gray scale values, color flow values, and angio or power Doppler information.

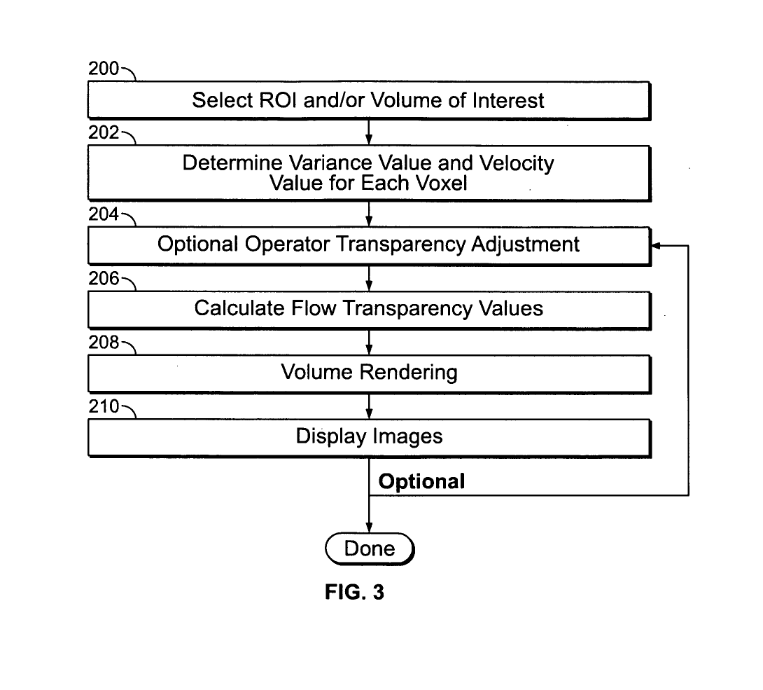

FIG. 3 illustrates a method for calculating flow transparency values for flow voxels within a volume of ultrasonic data. The calculation may be accomplished in real-time while scanning and acquiring ultrasonic data of a patient or on data which has been stored in a memory. In step 200, the operator selects a region of interest (ROI) which may include such anatomy as the mitral valve or the tricuspid valve. Optionally, the ROI may be the entire scanned volume of data. Each voxel represents blood flow or tissue, such as a point on an artery wall or a heart valve. Voxels representing blood flow are herein referred to as voxels or flow voxels, and voxels representing tissue are referred to as tissue voxels. Each flow voxel within the volume of data has associated parameters which may be used to describe and/or classify the voxel, such as variance, velocity, and amplitude. Velocity in color flow may also be referred to as frequency. Conventionally, variance is proportional to the square of the bandwidth of the Doppler signal, with unit frequency squared or velocity squared. Voxels that contain blood regions with large velocity gradients will show a large value of the variance parameter. Additionally, because of a physical mechanism referred to as the transit time effect, voxels representing regions with large velocities will also have a large variance. Therefore, velocity and variance are related such that a flow voxel having high velocity also has high variance. Variance may also be referred to as bandwidth or turbulence.

In step 202, the processor 116 determines a variance value and a velocity value for each voxel within the ROI. In step 204, the operator may optionally adjust a user defined transparency level which is used as an input to the transparency calculations. This adjustment may be accomplished manually through the user interface 120 by entering a variable input such as a number between zero and one. Alternatively, a user defined transparency level may be set by a protocol or be based on user preference. Typically, an input of zero indicates no transparency (fully opaque) and an input of one indicates full transparency. The user defined transparency level may be applied to change the flow transparency values for all voxels or for a portion of the voxels. For example, the user defined transparency level may not be applied above a predetermined threshold to prevent the removal of data representing high velocity blood flow.

In step 206, the processor 116 calculates the flow transparency value for each voxel with a transfer function which may be based on both velocity and variance using a continuum of threshold variance levels, such as flow transparency value equal to h(variance, velocity). By using a transfer function which includes the variance value, the voxels with high variance are displayed representative of actual flow velocity, eliminating display problems due to aliasing. In addition, the voxels that have high variance values may be emphasized on the display 118. For example, at a given velocity, the flow transparency may decrease as variance values increase. Optionally, the transfer function may calculate a portion or all of the flow transparency values based on an initial transparency level defined at least in part by the user defined transparency level.

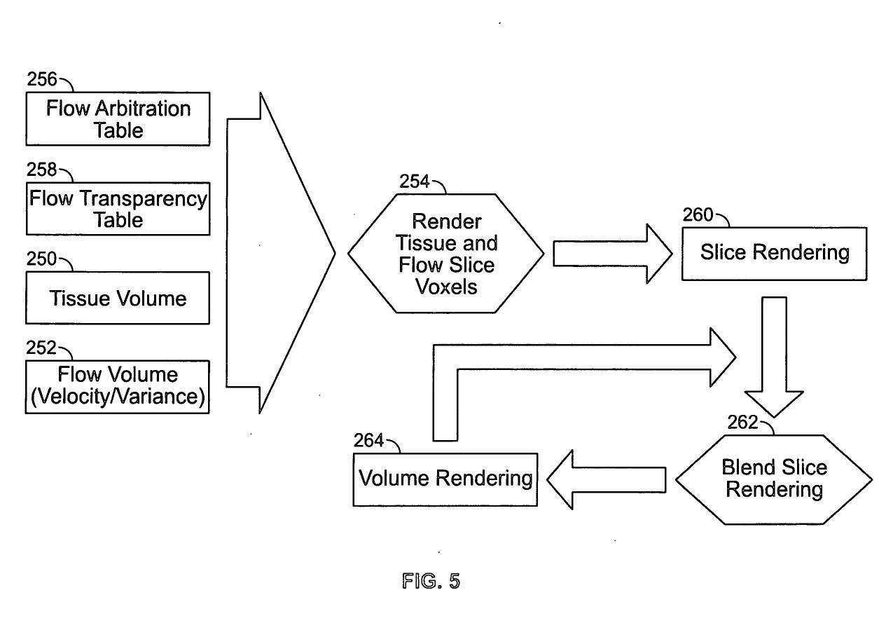

In step 208, volume rendering is accomplished using the flow transparency values calculated in step 206. FIG. 5 illustrates a flow chart of a method for flow volume rendering which may be accomplished by hardware and/or software as discussed previously with FIG. 2, or may also be accomplished by use of a graphics processor or graphics board. Also, other volume rendering methods, steps, and the like which are known in the art may be used.

The tissue volume and/or ROI identified in step 200 of FIG. 3 is input and split into multiple slices, such as by the volume scan converter 42. Voxels representing tissue volume 250 and flow volume 252 are identified. The flow volume 252 includes data such as the velocity and variance values for voxels representing blood flow.

A flow arbitration table 256 represents a predetermined transfer function or look-up table which calculates a flow arbitration value that is used to determine whether the flow voxel or tissue voxel is rendered. The flow arbitration table 256 may have one, two or more dimensions and utilize values such as velocity, variance, power, and the like. Other values may be used when forming the flow arbitration table 256. By way of example, the velocity and/or variance component(s) of the flow voxels in flow volume 252, as determined in step 202 of FIG. 3, may be used by the flow arbitration table 256 to calculate the flow arbitration value. The flow arbitration value and the intensity value of the tissue voxel may then be used to determine whether the flow voxel or the tissue voxel is rendered.

The corresponding flow voxels and tissue voxels are loaded as each slice is rendered 254. Each of the slices is rendered 260 by the volume rendering processor 46 and blended 262 with the result of the previous rendering step. A flow transparency table 258 may be stored in the flow transparency adjustment module 124 (FIG. 1) and accept the variable input from the operator through the user interface 120 and/or predefined and user defined transparency levels. The transparency values in the flow transparency table 258 are applied during the slice blend operation 262. Volume rendering 264 may combine all blended slices into a single rendering for display on the display 118.

Returning to FIG. 3, in step 210 the processor 116 displays the volume rendered image(s) on the display 118 based on at least the flow transparency values which correspond to displayed transparency levels. Optionally, the operator may change the user defined transparency level (step 204) to increase or decrease the level of transparency for some or all of the flow voxels. The processor 116 then calculates new transparency values (step 206), volume rendering is accomplished (step 208), and the image(s) are displayed (step 210).

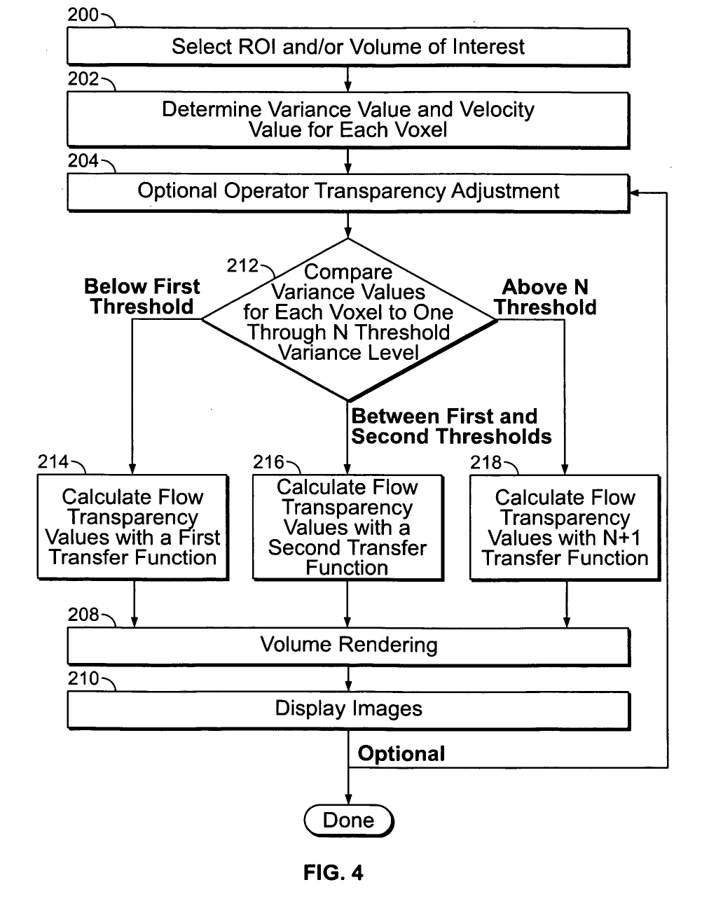

FIG. 4 illustrates an alternative method for calculating flow transparency values for flow voxels within a volume of ultrasonic data. The methods of FIGS. 3 and 4 share several steps which are indicated with like item numbers. In step 200, the operator selects a region of interest (ROI). In step 202, the processor 116 determines a variance value and a velocity value for each voxel within the ROI. In step 204, the operator may optionally adjust the user defined transparency level which is used as an input to the transparency calculations, or the processor 116 may access a preset transparency level value.

In step 212, the processor 116 compares the variance value for each voxel to one or more threshold variance levels. In one embodiment, if a single threshold variance level is used, the voxels are divided into two subsets of voxels wherein a first subset of voxels is below the threshold variance level and a second subset is above the threshold variance level. By way of example the threshold variance level may be a predetermined value above which values of Doppler variance are considered to be high. For example, the threshold variance level may be based on a fraction of the Nyquist velocity squared. Optionally, the operator may adjust and/or define one or more threshold variance levels.

In the example of FIG. 4, one through N threshold variance levels are used and thus the variance values are compared to first through N threshold variance levels. If the variance value for a particular voxel is below a first threshold variance level, the method passes to step 214. In step 214, the processor 116 calculates a flow transparency value for the voxel with a first transfer function. The first transfer function may base the flow transparency value on one or more parameters, such as the absolute value of the velocity value and the user defined transparency level. For example, a one-dimensional transfer function may utilize the velocity of the voxel to calculate the flow transparency value. A first voxel having a relatively lower velocity may have a higher transparency value and thus is more transparent compared to a second voxel that has a relatively higher velocity which results in a lower transparency value and a less transparent representation on the display 118.

Returning to step 212, if the variance value for the particular voxel is above the first threshold variance level and below a second threshold variance level, the method passes to step 216. The processor 116 calculates the flow transparency value for the voxel with a second transfer function which is different than the first transfer function. The processor 116 may calculate the flow transparency value with a one-dimensional transfer function by using, for example, the absolute value of the velocity, or with a two-dimensional transfer function by using both the variance value as well as the velocity value (or frequency) for the particular voxel, such as flow transparency value equal to h(variance, velocity).

Returning to step 212, if the variance value for the particular voxel is above N threshold variance level, the method passes to step 218. Additional threshold variance levels may be used between the second and N threshold variance levels. The processor 116 calculates the flow transparency value for the voxel with N+1 transfer function, which may be different from the first and second transfer functions, as well as any other intervening transfer functions.

In one embodiment, each of the transfer functions may produce a relatively flat curve, assigning each voxel having a variance value within a particular range of variance values to a similar or the same flow transparency value. Alternatively, the transfer function may produce a curve wherein voxels having a lower variance within the designated range are assigned a relatively higher flow transparency value and voxels having a higher variance are assigned a relatively lower flow transparency value.

In step 208, volume rendering is accomplished using the flow transparency values calculated in steps 214, 216 and 218. In step 210, the processor 116 displays the volume rendered image(s) on the display 118 based on at least the flow transparency values which correspond to display transparency levels. Optionally, the operator may input a different user defined transparency level (step 204). The processor 116 then calculates new transparency values (steps 214, 216 and 218), accomplishes volume rendering (step 208) and displays the image(s) (step 210).

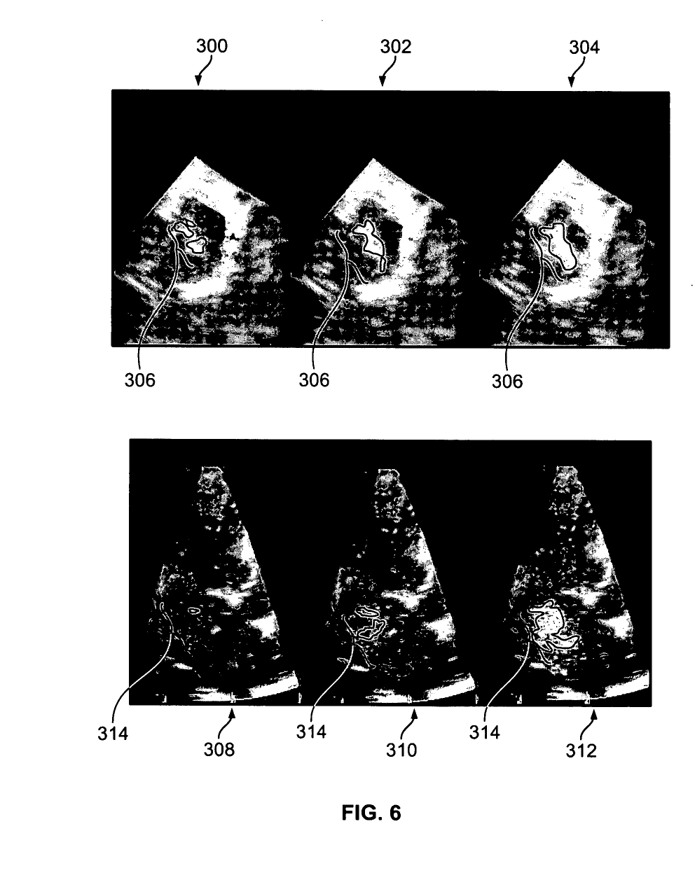

FIG. 6 illustrates ultrasound images of two different flow jets displayed with different levels of transparency. First, second and third images 300, 302 and 304 illustrate volume rendering of a first turbulent flow jet 306 at different levels of transparency control as displayed on display 118. When using color flow, the color is used to depict aspects of flow. Red and blue may indicate flow toward and away from the probe 106, while green indicates turbulence. In the first image 300, less transparency (such as a transparency of zero or near zero) is applied either automatically by the processor 116 or with the user defined transparency level. The first turbulent flow jet 306 is partially surrounded and obscured by low velocity flow in the atrium and/or ventricle, and thus less of the first turbulent flow jet 306 is displayed on the display 118. The correct size of the turbulent flow jet 306 cannot be seen or measured by the operator.

In the second image 302, the operator inputs a higher user defined transparency level (such as a value of 0.5) to apply a higher level of transparency to the flow transparency adjustment module 124. More of the low velocity blood flow is made transparent and thus more of the first turbulent flow jet 306 is displayed compared to the first image 300. In the third image 304, a respectively higher level of transparency is applied, such as a value of 1 or nearly 1, removing or making transparent most of the voxels displayed which indicate the low velocity blood flow and obscure the first turbulent flow jet 306.

Fourth, fifth and sixth images 308, 310 and 312 illustrate volume rendering of a second turbulent flow jet 314 at different stages of transparency control. With a low level of transparency applied in the fourth image 308, the second turbulent flow jet 314 is mostly obscured by the surrounding low velocity flow. The fifth image 310 has a higher level of transparency applied and more of the second turbulent flow jet 314 is visible compared to the fourth image 308. An even higher level of transparency is applied in the sixth image 312, and thus the second turbulent flow jet 314 is easily visualized by the operator.

A technical effect is using the variance data associated with ultrasonic data or voxels to calculate the transparency of the particular voxel on a display. In one embodiment, flow transparency values may be calculated for each voxel with a transfer function based on both velocity and variance using a continuum of threshold variance levels. In another embodiment, the flow transparency values may be calculated using one or more transfer functions associated with one or more threshold variance levels. The transfer functions may be different with respect to each other, and may utilize variance values and/or velocity values. The blood flow with high variance and/or high velocity is displayed with less transparency than blood flow with low variance and/or low velocity. An operator may change the displayed transparency level to adjust the transparency of all or a portion of the voxels.

While the invention has been described in terms of various specific embodiments, those skilled in the art will recognize that the invention can be practiced with modification within the spirit and scope of the claims.

What is claimed is:

1. A method for calculating flow transparency values for voxels representing blood flow within an ultrasonic volume of data, comprising:
identifying velocity values and variance values for voxels within a volume of data; and
calculating flow transparency values for the voxels based on a relationship between the variance value and the velocity value.

2. The method of claim 1, the voxels further comprising a first voxel, the calculating step further comprising:
comparing the variance value for the first voxel to a continuum of threshold variance levels; and
selecting a transfer function for calculating the flow transparency value for the first voxel based on a relationship between the variance value of the first voxel and the continuum of threshold variance levels.

3. The method of claim 1, the calculating step further comprising:
comparing the variance values for the voxels to a threshold variance level;
calculating the flow transparency values for the voxels having the variance values below the threshold variance level with a first transfer function; and
calculating the flow transparency values for the voxels having the variance values above the threshold variance level with at least one transfer function which is different than the first transfer function.

4. The method of claim 1, further comprising:
comparing the variance values for the voxels to at least one threshold variance level based on at least one of a predetermined frequency rate, a Nyquist frequency, and a variable input; and
calculating the flow transparency values based on a relationship of the variance values to the at least one threshold variance level.

5. The method of claim 1, further comprising accepting a user defined transparency level, the user defined transparency level being one of input by an operator and a predetermined value, the calculating step further comprising calculating the flow transparency values for the voxels based on the user defined transparency level.

6. The method of claim 1, the calculating step further comprising calculating the flow transparency values with at least one transfer function to display the voxels having relatively higher variance values with less transparency than the voxels having relatively lower variance values.

7. The method of claim 1, further comprising:
accepting a user defined transparency level; and
calculating at least a portion of the flow transparency values based on the user defined transparency level.

8. An apparatus for displaying blood flow within a volume of ultrasonic data, comprising:
a processor for identifying a variance value and a velocity value for each voxel within a volume of data comprising blood flow, the processor calculating a flow transparency value for each of the voxels based on a relationship between the variance value and the velocity value;
a volume rendering processor utilizing the flow transparency values while volume rendering the volume of data; and
a display for displaying volume rendered data based on the flow transparency values.

9. The apparatus of claim 8, the processor comparing the variance value for a first voxel to a continuum of threshold variance levels, the processor selecting a transfer function for calculating the flow transparency value for the first voxel based on a relationship between the variance value of the first voxel and the continuum of threshold variance levels.

10. The apparatus of claim 8, the processor comparing a first variance value for a first voxel to N threshold variance levels, the processor calculating the flow transparency value for the first voxel with a transfer function based on the relationship of the first variance value to the N threshold variance levels.

11. The apparatus of claim 8, further comprising:
a user interface; and
a flow transparency adjustment module accepting a user defined transparency level from the user interface, the processor further calculating the flow transparency value for at least a portion of the voxels based on the user defined transparency level.

12. The apparatus of claim 8, the processor further comprising calculating the flow transparency values with at least one transfer function to produce flow transparency values for displaying the voxels having a relatively higher variance value with less transparency on the display compared to the voxels having a relatively lower variance value.

13. The apparatus of claim 8, further comprising defining a threshold variance level based on at least one of a predetermined frequency rate, a Nyquist frequency and a user determined input value, the display displaying the voxels having the variance value above the threshold variance level with less transparency compared to the voxels having the variance value below the threshold variance level.

14. A method for calculating flow transparency values for voxels representing blood flow within an ultrasonic volume of data, comprising:
identifying a variance value for each of the voxels within a volume of data;
comparing the variance value for each of the voxels to a continuum of threshold variance levels; and
calculating a flow transparency value for each of the voxels with a transfer function based on at least the variance value and a relationship between the variance value and the continuum of threshold variance levels.

15. The method of claim 14, further comprising identifying a velocity value for each of the voxels, the transfer function further calculating the flow transparency value based on at least the velocity value.

16. The method of claim 14, further comprising:
inputting a variable input; and
adjusting the flow transparency value based on the variable input.

17. The method of claim 14, further comprising volume rendering the volume of data utilizing the flow transparency values.

18. The method of claim 14, the continuum of threshold variance levels further comprising at least a first threshold variance level, the method further comprising:
calculating the flow transparency value of the voxels having the variance value above the first threshold variance level with a first transfer function; and
calculating the flow transparency value of the voxels having the variance value below the first threshold variance level with a second transfer function different from the first transfer function.

19. The method of claim 14, further comprising choosing the transfer function from a plurality of transfer functions based on the relationship between the variance value and the continuum of threshold variance levels.

20. The method of claim 14, further comprising volume rendering the volume of data, the volume rendering step further comprising a slice blend operation for blending rendered slices, the slice blending operation utilizing the flow transparency values.
